(19) United States
(12) Patent Application Publication
(10) Pub. No.: US 2026/0027186 A1
(43) Pub. Date: Jan. 29, 2026

(54) SYNTHETIC-BASED HYDROGELS, SYNTHETIC-BASED HYDROGEL COMPOSITES AND METHOD FOR ENTEROSORPTIVE REMOVAL OF TARGET MOLECULES

(71) Applicant: University of Kentucky Research Foundation, Lexington, KY (US)

(72) Inventors: Mei Li Weatherly, Lexington, KY (US); Ifrah Hammad, Lexington, KY (US); Sachin Sundar, Lexington, KY (US); James Zach Hilt, Lexington, KY (US)

(21) Appl. No.: 19/281,243

(22) Filed: Jul. 25, 2025

Related U.S. Application Data
(60) Provisional application No. 63/675,417, filed on Jul. 25, 2024.

(51) Int. Cl. A61K 38/38; A61K 9/06; A61K 38/17; A61K 47/10; A61K 47/34

(52) U.S. Cl. CPC A61K 38/385; A61K 9/06; A61K 38/177; A61K 47/10; A61K 47/34

(57) ABSTRACT

A method for enterosorptive removal of a target molecule from a digestive tract of an individual, includes orally administering to the individual a synthetic-based hydrogel or synthetic-based hydrogel composite incorporating a hydrogel base material and a functional co-monomer. The synthetic-based hydrogel or synthetic-based hydrogel composite is adapted to bind and remove the target molecule from the digestive track without being metabolized or absorbed into systemic circulation.

### RELATED APPLICATIONS

This application claims the benefit of U.S. Provisional Patent Application Ser. No. 63/675,417, filed on Jul. 25, 2024, the full disclosure of which is incorporated herein by reference.

### TECHNICAL FIELD

This document relates generally to the field of enterosorption and, more particularly to synthetic-based hydrogels and related methods adapted for binding and removing harmful target molecules from the digestive tract of an individual without being metabolized or absorbed into the systemic circulation and thereby excreted unchanged with the absorbed target molecules.

### BACKGROUND

Carcinogens refer to a large and ever-growing group of substances which cause cancer. In general, carcinogens only truly increase the risk of cancer and not all people exposed to carcinogens will develop this disease. Individual factors such as length and intensity of exposure, as well as genetic disposition, can influence the ultimate outcome. At the heart of this mechanism lies DNA damage which can occur via DNA strand breaks, formation of DNA adducts, lesions, or DNA-protein cross-links among others. The individual physio-chemical properties of a particular carcinogen impact the mechanism of DNA damage, which depends on its classification as a physical or chemical cancer-causing agent.

It's important to keep in mind that carcinogens themselves can be dangerous, but exposure to their precursors can also pose quite a health risk. One prime example of this is with nitrates. These are naturally occurring in some plants and in water but are found in especially elevated levels in some cases in part due to the use of fertilizers and fertilizer runoff, as well as growth conditions. Nitrates are also a common food additive meant to help preserve various meats and cheeses, and some have even set limits on these, including the European Union and the World Health Organization. Unfortunately in some cases leafy vegetables and fruit contain some of the highest nitrate levels and this can be harder to regulate.

The problem with nitrates is what happens when they enter the body. In general, up to around 75% of ingested nitrates are excreted from the body, but up to 90% of the nitrates in the human body are actually a result of converted nitrates. Once recycled, nitrates can often reach concentrations in the salivary glands that are ten times higher than in the plasma. Ultimately, around 5% of the nitrate intake is converted to nitrates by bacteria found within the oral cavity, at which point much of this is converted to nitric oxide in the stomachs acidic environment. Although nitric oxide has its own benefits, including protecting the cardiovascular system (increased blood flow, relaxed blood vessels, etc.) nitrates may undergo other reactions as well. For example, when nitrates react with amines, they can form nitrosamines, which are classified as N-nitroso compounds. Primary amines are responsible for the formation of unstable nitrosamines which are prone to degradation, and tertiary amines are unreactive, but secondary amines are responsible for the formation of a group of nitrosamines which have been found to be carcinogenic. In order for the nitrosamines to exhibit their carcinogenic behaviors, they have to be metabolically activated (often by cytochrome P450 enzymes). Their mechanism of action is as an alkylating agent, meaning that they attach alkyl groups to DNA such DNA adducts form, which can cause mutations and therefore damage the DNA. Connections have been made between nitrate intake and breast, gastric, colorectal, esophageal, and thyroid cancer among others.

Consider that acceptable daily intake (ADI) levels for nitrates and nitrites have been set and a study about Swedish children actually found that while nitrite intake levels for a 4-12 year old children was within range, the added nitrites from converted nitrites pushed 12% of the 4 year olds over this threshold. Foods containing nitrates are widespread and quite common for many people, and this make their regulation and avoidance much more difficult.

Per- and poly-fluoroalkyl substances (PFAS) pose documented and suspected health problems to humans, such as abnormal fetal development, increased risk of cancer, immunosuppression, and thyroid dysfunction. Due to prevalent concentrations of PFAS in local drinking water, various consumer products, and other commonplace items, removal of PFAS from the body is of interest to the EPA. Current PFAS treatment methods include anion exchange, reverse osmosis, and nanofiltration; however, none of these methods are suitable for in vivo removal of PFAS.

Synthetic food dyes, specifically FD&C food dyes, are a class of FDA-approved food dyes from non-naturally derived sources such as petroleum and coal tar. Currently, there are seven FD&C straight food dyes: Red #40, Red #3, Yellow #5, Yellow #6, Green #3, Blue #1, Blue #2. While research is inconclusive, the synthetic food dyes are suggested to cause multiple adverse health effects. For example, Red #40 is strongly suggested to be carcinogenic, Yellow #5 and Yellow #6 are linked to inducing allergenic reactions in patients who have urticaria and other chronic allergy predispositions, and Blue #1 and Blue #2 are linked to attention deficit and other developmental-behavioral issues. Despite these adverse health effects, lobbyist support for synthetic food dyes remains strong, and although other countries have tentatively restricted or outright banned the use of FD&C dyes, the United States has yet to follow suit due to increasing corporate pressure to allow FD&C dyes due to the psychological propensity of vibrant coloring to induce hunger. While the FDA recently banned the use of Red #3, following many of its European predecessors, other petitions to ban synthetic food dyes have not been addressed.

Due to the multifaceted nature of (a) carcinogens, (b) carcinogen precursors, (c) nitrosamines, (d) nitrates, (e) PFAS and (f) synthetic food dyes, outright bans and successful removal from mass consumption are difficult and tedious tasks hindered by lobbying, consumerism, and their substantial prevalence in consumables. As a result, an in vivo method for their removal from the body, one that directly mitigates the health impacts of their accumulation and presence, is needed. This document addresses that need.

Enterosorption is defined as an oral adsorbent that is used to bind and remove contaminants from the digestive tract without being metabolized or absorbed into systemic circulation themselves, thus being excreted unchanged with the adsorbed contaminant. In literature, enterosorption has been used for the removal of mycotoxins, heavy metal toxins, and endo/exo-toxins.

### SUMMARY

Each of the following terms written in singular grammatical form: “a”, “an”, and “the”, as used herein, means “at least one”, or “one or more”. Use of the phrase “One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or, unless the context clearly dictates otherwise. For example, the phrase: “an additive”, as used herein, may also refer to, and encompass, a plurality of additives.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The phrase “consisting of”, as used herein, is closed-ended and excludes any element, step, or ingredient not specifically mentioned. The phrase “consisting essentially of”, as used herein, is a semi-closed term indicating that an item is limited to the components specified and those that do not materially affect the basic and novel characteristic(s) of what is specified.

Terms of approximation, such as the terms about, substantially, approximately, etc., as used herein, refers to ±10% of the stated numerical value.

In accordance with the purposes and benefits set forth herein, a method for enterosorptive removal of a target molecule from a digestive tract of an individual, comprises, consists of or consists essentially of orally administering to the patient a synthetic-based hydrogel or synthetic-based hydrogel composite incorporating a hydrogel base material and a functional co-monomer, wherein the synthetic-based hydrogel or synthetic-based hydrogel composite is adapted to bind and remove the target molecule from the digestive track without being metabolized or absorbed into systemic circulation.

The hydrogel base material may be selected from a first group of materials consisting of poly(ethylene glycol), poly(acrylamide), poly(hydroxyethyl methacrylate), poly(acrylic acid), poly(methacrylic acid), poly(N-isopropyl acrylamide), poly(vinyl alcohol), and combinations thereof. The functional co-monomer may be selected from a second group of materials consisting of a co-monomer with cationic functionalities, a co-monomer with anionic functionalities, a co-monomer with Zwitterionic functionalities, a co-monomer with aromatic functionalities, a co-monomer with hydrophobic functionalities and combinations thereof.

The composite additive may be selected from a third group of materials consisting of a hydrophobic particulate, a hydrophobic polymer, an ionic particulate, a biological polymer, an ionic polymer, a cationic particulate, a cationic polymer, an anionic particulate, an anionic polymer, a Zwitterionic polymer, a Zwitterionic particulate, a protein, a carbohydrate, deoxyribonucleic acid (DNA), ribonucleic acid (RNA), and combinations thereof.

The composite additive may also be selected from a third group of materials consisting of an activated carbon, a powdered activated carbon, a granular activated carbon, an extruded activated carbon, an impregnated activated carbon, and combinations thereof. Still further, the composite additive may be selected from a third group of polymer materials consisting of poly(styrene), poly(methyl methacrylate), poly(ethylene), polypropylene, poly(vinyl chloride), polytetrafluoroethylene, poly(dimethylsiloxane), polyester, polyurethane, poly(vinylidene fluoride), a fluoropolymer, and combinations thereof. Still further, the composite additive may be selected from a third group of polymer materials consisting of poly(lysine), poly(ethyleneimine), chitosan, cationic cellulose, poly(acrylic acid), poly(methacrylic acid) and combinations thereof. In addition, the composite additive may be selected from a third group of materials consisting of a lectin, a DNA binding protein, an RNA binding protein, an albumin, a carbohydrate, and combinations thereof.

In at least some of the many possible embodiments, the method includes binding and removing (a) carcinogens, (b) carcinogen precursors, (c) nitrosamines, (d) nitrates or (e) combinations thereof from the digestive track. In some embodiments, the method includes
binding and removing a per-fluoroalkyl substance or a poly-fluoroalkyl substance from the digestive track. In still other embodiments, the method includes binding and removing a dye or a synthetic food dye from the digestive track.

The method may include orally administering the hydrogel composite at a dosage rate of between 0.002 to 1.0 grams at a time interval of between every four hours up to once a month.

In one particularly useful embodiment, the method includes using poly(ethylene glycol) diacrylate (PEGDA) as the hydrogel base material, using (3-acrylamidopropyl)trimethylammonium chloride (DMAPA-Q) as the functional co-monomer, and using activated carbon as the composite additive.

In accordance with an additional aspect, a hydrogel system, comprises, consists of or consists essentially of a synthetic-based hydrogel or synthetic-based hydrogel composite incorporating a hydrogel base material and a functional monomer, wherein the synthetic-based hydrogel or synthetic-based hydrogel composite is adapted to bind and remove the target molecule from the digestive track without being metabolized or absorbed into systemic circulation.

As noted above, the hydrogel base material may be selected from a first group of materials consisting of poly(ethylene glycol), poly(acrylamide), poly(hydroxyethyl methacrylate), poly(acrylic acid), poly(methacrylic acid), poly(N-isopropyl acrylamide) and combinations thereof. The functional co-monomer may be selected from a second group of materials consisting of a co-monomer with cationic functionalities, a co-monomer with anionic functionalities, a co-monomer with Zwitterionic functionalities, a co-monomer with aromatic functionalities, a co-monomer with hydrophobic functionalities, and combinations thereof.

The composite additive may be selected from a third group of materials consisting of a hydrophobic particulate, a hydrophobic polymer, an ionic particulate, a biological polymer, an ionic polymer, a cationic particulate, a cationic polymer, an anionic particulate, an anionic polymer, a Zwitterionic polymer, a Zwitterionic particulate, a protein, a carbohydrate, deoxyribonucleic acid (DNA), ribonucleic acid (RNA), and combinations thereof.

More specifically, the composite additive may be selected from a third group of materials consisting of an activated carbon, a powdered activated carbon, a granular activated carbon, an extruded activated carbon, an impregnated activated carbon, and combinations thereof. The composite additive may also be selected from a third group of polymer materials consisting of poly(styrene), poly(methyl methacrylate), poly(ethylene), polypropylene, poly(vinyl chloride), polytetrafluoroethylene, poly(dimethylsiloxane), polyester, polyurethane, poly(vinylidene fluoride), a fluoropolymer, and combinations thereof. Still further, the composite additive may also be selected from a third group of polymer materials consisting of poly(lysine), poly(ethyleneimine), chitosan, cationic cellulose, poly(acrylic acid), poly(methacrylic acid) and combinations thereof. Still further, the composite additive may also be selected from a third group of materials consisting of a lectin, a DNA binding protein, an RNA binding protein, an albumin, a carbohydrate, and combinations thereof.

The hydrogel system may further include a weight percentage of additive of between about 0.1% and 50% in the synthetic-based hydrogel composite.

In the following description, there are shown and described several embodiments of the hydrogel system and method for enterosorptive removal of a target molecule. As it should be realized, the hydrogel system and method are capable of other, different embodiments and its several details are capable of modification in various, obvious aspects all without departing from the hydrogel system and method as set forth and described in the following claims. Accordingly, the drawing figures and descriptions should be regarded as illustrative in nature and not as restrictive.

### BRIEF DESCRIPTION OF THE DRAWING FIGURES

The accompanying figures incorporated herein and forming a part of the specification, illustrates certain aspects of the new and improved method and together with the description serves to explain certain principles thereof. A person of ordinary skill in the art will readily recognize from the following discussion that alternative embodiments of the method may be employed without departing from the principles described below.

FIG. 1 illustrates the nomenclature for the hydrogels discussed below in the “Experimental” section of this document.

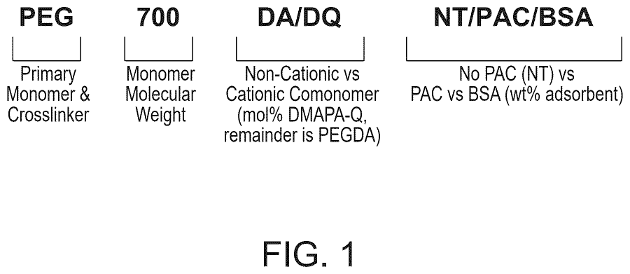

FIG. 2 is a schematic illustration of the procedure used to form the hydrogel composites.

FIG. 3 is a schematic illustration of the procedure used to cut, wash and crush the hydrogels into a powder.

FIG. 4 is a schematic illustration of the inverse swelling procedure.

FIG. 5 is a schematic illustration of the indigo carmine and metanil yellow sorption study procedure.

FIG. 6 is a graph illustrating FTIR results for various NT systems (systems with no powdered activated carbon).

FIG. 7 is a graph illustrating FTIR results for various powdered activated carbon systems.

FIG. 8 is a graph illustrating FTIR results for various bovine serum albumin (BSA) systems.

FIG. 9 is a graph of FTIR results for bovine serum albumin.

FIG. 10 is a graph of FTIR results for bovine serum albumin systems with modified synthesis procedures.

FIG. 11 is a plot illustrating inverse swelling results for bovine serum albumin hydrogels.

FIG. 12 is a graph of absorbance versus concentration for 0.1 mM indigo carmine calibration curve.

FIG. 13 is a plot of 0.1 mM indigo carmine sorption study, 0.5 mg/mL, 20 hours.

FIG. 14 is a plot of percent removal for various hydrogels for 0.2 mM indigo carmine sorption study, 0.5 mg/mL, 20 hours.

FIG. 15 is a plot of percent removal for various hydrogels for 0.4 mg/mL, 46 hours.

FIG. 16 is a plot of percent removal for various hydrogels for 0.8 mM indigo carmine sorption study, 0.5 mg/ml, 20 hours.

FIG. 17 is a plot of removal efficiency for various hydrogels for 0.8 mM indigo carmine kinetic sorption study, 0.5 mg/mL, 2-48 hours.

FIG. 18 is a plot of percent removal for 0.8 mM indigo carmine sorption study, 0.5 mg/mL, 48 hours.

FIG. 19 is a plot of percent removal for various hydrogels for 0.8 mM metanil yellow sorption study, 0.5 mg/mL, 20 hours.

FIG. 20 is a plot of percent removal for various hydrogels for 0.5 mM indigo carmine sorption study, 0.5 mg/mL, 20 hours.

FIG. 21 is a plot of percent removal for various hydrogels for 0.8 metanil yellow sorption study, 0.5 mg/mL, 20 hours.

FIG. 22 is a plot for 0.8 mM indigo carmine sorption study, 0.5 mg/mL, 20 hours.

FIG. 23 is a plot of percent removal for various hydrogels for 0.8 metanil yellow sorption study, 0.5 mg/mL, 20 hours.

Reference will now be made in detail to the present preferred embodiments of the method for enterosorptive removal of a target molecule.

### DETAILED DESCRIPTION

A method for enterosorptive removal of a target molecule from a digestive tract of an individual, may be broadly described as including orally administering to the patient a synthetic-based hydrogel or synthetic-based hydrogel composite incorporating a hydrogel base material and a functional co-monomer, wherein the synthetic-based hydrogel or synthetic-based hydrogel composite is adapted to bind and remove the target molecule from the digestive track without being metabolized or absorbed into systemic circulation. For purposes of this document, the terminology “synthetic-based hydrogel” refers to a covalently crosslinked hydrogel requiring a chemical reaction between monomers and crosslinkers as a hydrogel base material with the addition of at least one functional co-monomer. The terminology “synthetic-based hydrogel composite” refers to a synthetic-based hydrogel incorporating an additive of the type described elsewhere in this document that is selected to bind and hold the target molecule.

The hydrogel base material may be selected from a first group of materials consisting of, but not necessarily limited to, poly(ethylene glycol), poly(acrylamide), poly(hydroxyethyl methacrylate), poly(acrylic acid), poly(methacrylic acid), poly(N-isopropyl acrylamide) and combinations thereof.

The functional co-monomer may be selected from substantially any co-monomer with cationic functionalities, any co-monomer with anionic functionalities, any co-monomer with Zwitterionic functionalities, any co-monomer with aromatic functionalities, any co-monomer with hydrophobic functionalities and combinations thereof.

Cationic monomers include, but are not necessarily limited to: 2-(N,N-Diethylamino)ethyl methacrylate (DEAEMA), 2-(N,N-Dimethylamino)ethyl acrylate (DMAEA), 2-(N,N-Dimethylamino)ethyl methacrylate (DMAEMA), 2-(tert-Butylamino)ethyl methacrylate (TBAEMA), 2-Acryloxyethyltrimethylammonium chloride (AETMAC), 2-Aminoethyl Methacrylate Hydrochloride (AEMA HCl), 2-Diisopropylaminoethyl methacrylate (DPAEMA), and 2-N-Morpholinoethyl acrylate (MEA-Morph).

Anionic monomers include, but are not necessarily limited to acrylic acid (AA), methacrylic acid (MAA), 2-Sulfoethyl methacrylate (SEMA), 3-Sulfopropyl acrylate, potassium salt (SPA-K), 3-Sulfopropyl methacrylate, potassium salt (SPMA-K), Beta-Carboxyethyl Acrylate (BETA-C), and 2-(methacryloxy)ethyl phosphate.

Zwitterionic monomers include, but are not necessarily limited to, 3-Sulfopropyldimethyl-3-methacrylamidopropylammonium, inner salt (SPDMA-IS) and Methacryloyl-L-Lysine. Aromatic monomers include, but are not necessarily limited to, Styrene, 2-Phenoxyethyl Methacrylate (POEMA), 2-Phenylethyl Acrylate (PEA), 2-Phenylethyl methacrylate (PEMA), and Benzyl acrylate (BA). Hydrophobic monomers include, but are not necessarily limited to, Ethylene, Methyl methacrylate, 2-n-Butoxyethyl methacrylate (BEM), and 3,3,5-Trimethylcyclohexyl methacrylate (TMCHMA).

The additive may be selected from a list of additives consisting of a hydrophobic particulate, a hydrophobic polymer, an ionic particulate, a biological polymer, an ionic polymer, a cationic particulate, a cationic polymer, an anionic particulate, an anionic polymer, a Zwitterionic polymer, a Zwitterionic particulate, a protein, a carbohydrate, deoxyribonucleic acid (DNA), ribonucleic acid (RNA), and combinations thereof.

The terminology “biological polymer” refers broadly to natural polymers produced by cells of living organisms, including plants, animals, bacteria and fungi. The three main classes of biological polymers include polynucleotides, polypeptides and polysaccharides. Ribonucleic acid (RNA) and deoxyribonucleic acid (DNA) are examples of long polymers of nucleotides. Polypeptides include proteins and shorter polymers of amino acids (e.g. collagen, actin and fibrin). Polysaccharides are linear or branched chains of sugar carbohydrates (e.g. starch, cellulose and alginate). Other examples of biological polymers include, but are not necessarily limited to natural rubber, suberin, lignin, cutin, cutan, melanin, and polyhydroxyalkanoates.

Useful additives include, but are not necessarily limited to: (a) activated carbon, a powdered activated carbon, a granular activated carbon, an extruded activated carbon (in its smallest size range of around or below 1.0 mm diameter), an impregnated activated carbon, (b) polymers consisting of poly(styrene), poly(methyl methacrylate), poly(ethylene), polypropylene, poly(vinyl chloride), polytetrafluoroethylene, poly(dimethylsiloxane), polyester, polyurethane, poly(vinylidene fluoride), a fluoropolymer, poly(lysine), poly(ethyleneimine), chitosan, cationic cellulose, poly(acrylic acid), poly(methacrylic acid), and (c) additives, such as, a lectin, a DNA binding protein, an RNA binding protein, an albumin, a carbohydrate. Any combination of the above listed additives may also be used.

In accordance with one aspect, the synthetic-based hydrogels and synthetic-based hydrogel composites are particularly useful for binding and removing (a) carcinogens, (b) carcinogen precursors, (c) nitrosamines, (d) nitrates or (e) combinations thereof from the digestive tract. In accordance with another aspect, the synthetic-based hydrogels and synthetic-based hydrogel composites are useful for binding and removing per-fluoroalkyl substances, poly-fluoroalkyl substances, dyes and synthetic food dues, including Red #40, Red #3, Yellow #5, Yellow #6, Green #3, Blue #1, Blue #2 from the digestive tract of an individual. For purposes of this document, “patient” refers to humans, mammals, domestic farm animals, and other animals including a digestive tract.

The synthetic-based hydrogel or synthetic-based hydrogel composite may be administered to the patient at a dosage rate of between 0.002 to 1.0 grams over a period of every four hours, up to and including every month. Alternative dosage rates include, but are not necessarily limited to: between 0.002 and 0.8 grams, between 0.002 and 0.7 grams, between 0.002 and 0.5 grams, between 0.002 and 0.3 grams, between 0.002 and 0.2 grams, between 0.005 and 1 gram, between 0.005 and 0.8 grams, between 0.005 and 0.5 grams, between. 1 and 1 gram, between. 1 and 0.8 grams, between 0.1 and 0.5 grams, between 0.3 and 1 gram, between 0.3 and 0.8 grams, between 0.3 and 0.5 grams, between 0.5 and 1 gram, and between 0.5 and 0.8 grams. Alternative time periods for each dose may also vary to a period of between every four hours and every week, between every 8 hours and every month, between every eight hours and every week, between every 12 hours and every month, between every 12 hours and every week, between every day and every month, between every day and every week, and between every 24 hours and every 48 hours. The dosage for treatment with the synthetic-based hydrogels and hydrogel composites depends on such factors as the age, weight and condition of the individual as well as the condition being treated. The compound may be administered before meals for maximum effect to be obtained.

The weight percentage of additive added to the synthetic-based hydrogel composites may be between 0.1% and 50%. In other embodiments, the weight percentage of additive added to the hydrogel composites may be between 0.1% and 20%. In still other embodiments, the weight percentage of additive added to the hydrogel composites may be between 0.5% and 50%, between 0.5% and 20%, between 1.0% and 50%, between 1.0% and 20%, between 5% and 50%, between 5% and 20%, between 10% and 50%, between 10% and 20%, between 25% and 50%, between 25% and 40% and between 30% and 40%.

The synthetic-based hydrogel or synthetic-based hydrogel composites may be made by any appropriate method for making hydrogels and hydrogel composites known to those skilled in the art. Thus, the hydrogels may be synthesized by combining the desired proportions of (a) monomer or monomers and/or crosslinker for the hydrogel base material and at least one functional co-monomer to (b) deionized (DI) water or other appropriate solvent that provides mutual solubility at various w/w concentrations in a borosilicate scintillation vial. For composite hydrogels, the desired additive or combination of additives is added to the deionized water alongside the hydrogel base material monomer or monomers and/or crosslinker and functional co-monomer. The solution is stirred and the initiator and catalyst are simultaneously added to the mixture and stirred briefly to initiate free-radical polymerization in which the catalyst splits the initiator in half to form a pair of radicals. The gels may then be cast in a 48-well polystyrene plate in appropriate volume increments using an Eppendorf positive displacement pipette or other appropriate means known to those skilled in the art. A uniform metallic cylindrical cutting tool may be used to extract the hydrogels and hydrogel composites. All gels may then be washed in DI water. The gels may then be freeze-dried to preserve expansibility.

Typically, the synthetic-based hydrogel or synthetic-based hydrogel composite is prepared as a cylinder plug within a microplate well, but it should be appreciated that it may be cast into almost any shape. These are the bulk structures as prepared. These may then be crushed and ground into a powder using various techniques known to those skilled in the art to produce powders of desired particle size and size distributions. In the dry powder form, these hydrogels and hydrogel composites may be added to various types of formulations to produce tablets, capsules, gummies and the like for oral administration to an individual.

If desired, the dry powder form of the synthetic-based hydrogels and synthetic-based hydrogel composites may be combined with any useful excipients of a type known in the art. Useful excipients include, but are not limited to, known binders, diluents, fillers, glidants, antioxidants, preservatives, sweeteners, stabilizing agents, coating agents and flavoring agents. Suitable excipients are substances that do not react with the hydrogel powder and any composite additives. Thus, the tablet or capsule may contain, for example, gum, starch, gelatin or even a buffer used in quantities or proportions useful for desired purposes as known to those skilled in the art. In the simplest iteration, the dried hydrogel powder is directly added to the capsule which is then sealed.

### EXPERIMENTAL

### Materials

Poly(ethylene glycol) diacrylate Mn 700 (PEGDA 700) (CAS No. 26570-89-9), ammonium persulfate (APS) (CAS No. 7727-54-0), indigo carmine (CAS No. 860-22-0), and metanil yellow (CAS No. 587-98-4) were purchased from Sigma-Aldrich. Carbon powder, activated, Norit GSX, steam activated, acid washed (CAS No. 7440-44-0) was purchased from Alfa Aesar. (3-Acrylamidopropyl)-trimethylammonium chloride (DMAPA-Q) (CAS No. 45021-77-0) and tetramethylethylenediamine (TEMED) (CAS No. 110-18-9) were purchased from TCI Chemicals. Albumin, bovine (CAS No. 9048-46-8) (BSA) was purchased from VWR.

### Synthesizing Polymers

All the polymer systems were formed using free radical polymerization. PEGDA is a monomer that has been functionalized such that it can act as its own crosslinker due to its acrylate group on either end of the molecule. DMAPA-Q, on the other hand, is a cationic monomer that can only form crosslinks on one end due to its single C═C double bond. The specific radical initiator and catalyst combination that was used was APS and TEMED.

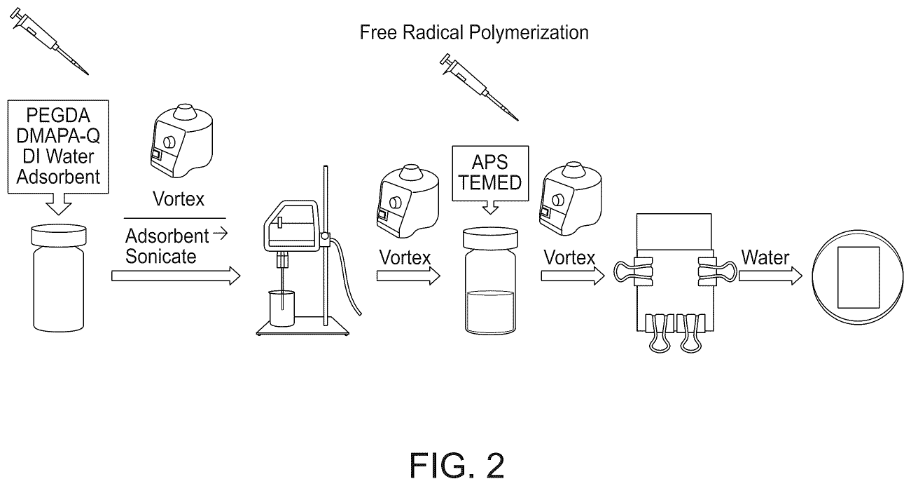

To prepare the various polymers, the first step was to make the 10 wt % APS solution by weighing out solid APS and adding DI water in the correct proportions to a 20 mL scintillation vial (or a 1.5 mL microcentrifuge tube depending on the volume of APS needed) and then vortexing until completely dissolved (a new APS solution was made each day synthesis was performed). See FIG. 2. Note that the 10 wt % APS stock solution was added at a different wt % to the monomer mixture, depending on the presence of DMAPA-Q, PAC and BSA, and the TEMED was added at ⅙ of that volume. Depending on the exact recipe, the PEGDA and DMAPA-Q were then added to a separate 20 mL scintillation vial using a repeater pipette—along with DI water as the solvent of choice—and finally PAC or BSA was weighed out on a scale and added as well.

Next, the components were mixed using a vortex. If PAC was added, the mixture was also sonicated using a Model 500 Sonic Dismembrator (Fisher Scientific) which uses sound energy to physically disperse and incorporate the PAC into the polymer network. For the hydrogel systems with BSA, the mixture was not always sonicated. During this process, the vial was clamped into place and placed in an ice bath to help maintain the mixture's temperature at a reasonable level. Sonication was performed using a 5 seconds on/10 seconds off pulsing pattern for a total pulsing time of 1 minute and at an amplitude of 31%.

The final step was to add the APS (initiator) and TEMED (catalyst) simultaneously to the vial and to vortex very briefly to initiate free-radical polymerization in which the TEMED splits the APS in half to form a pair of radicals. To create a polymer sheet of uniform thickness for ease of characterization, the polymer was immediately transferred to a device (polymer pal) using a sterile transfer pipette in which two pieces of glass are clamped together with binder clips with a gap in the middle. As this polymerization reaction is exothermic, the glass became hot, and the reaction was deemed complete upon subsequent cooling and/or the material's apparent loss of fluidity. The polymerization reactions were relatively quick with the NT systems taking longer to form than the PAC systems. These sheets of polymer were then placed in petri dishes containing DI water and allowed to sit overnight.

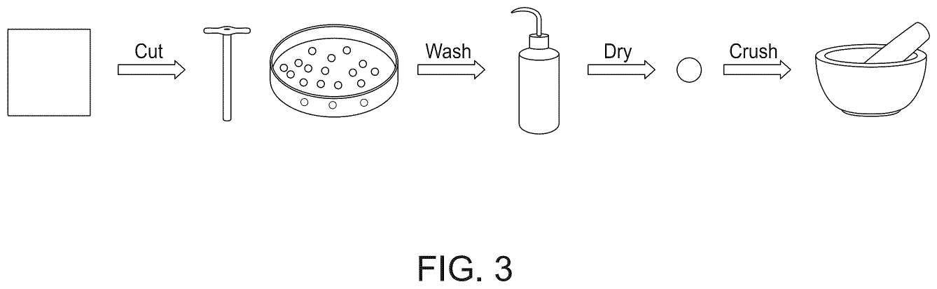

The next day or two was spent cutting the polymers. See FIG. 3. This is when the water was drained from the dish and a small metal tool was used to cut the polymer into uniform circular discs (analogous to a cookie cutter). Excess polymer was disposed of and then the polymer discs were flushed with DI water multiple times to wash away unreacted material which was removed using a transfer pipette until the water added to the petri dish was clear. To prepare for characterization and sorption studies, the polymer discs were dried in an oven (around 75° C. except for one batch of polymers) and often crushed into a powder using a mortar and pestle.

The polymer recipes used over the course of this research are contained in Table 1. This groups the polymers into 4 categories, depending on their incorporation of cationic co-monomer and adsorbent. It then lists out the amount of each monomer either as a volume or millimole basis (DMAPA-Q incorporation is expressed as a mole % relative to the total amount of monomer used). All water was added at a 1:1 w/w % with the monomers. Adsorbent was added at 1, 5, or 10 wt % of the monomer mixture. Finally, APS was added over a range of wt % and TEMED was added at ⅙ of that volume. Note that these are only feed amounts and that there may still be unreacted polymer that is washed away.

TABLE 1
Polymer recipes indicating the amount of PEGDA 700, DMAPA-Q, Water,
Adsorbent, APS, and TEMED grouped according to the hydrogel system

| | PEGDA | | | | | |
|---|---|---|---|---|---|---|
| System | 700 | DMAPA-Q | Water | Adsorbent | APS | TEMED |
| Non-cationic | 2 mL | — | 1:1 | — | 0.5-1 | APS/6 |
| No adsorbent | | | | | wt % | |
| Non-cationic | 2 mL | — | 1:1 | 1, 5, 10 | 0.5-1 | APS/6 |
| Adsorbent | | | | wt % | wt % | |
| Cationic | 7.5 mmol | 7.5 mmol | 1:1 | — | 0.5-1 | APS/6 |
| No adsorbent | basis | basis | | | wt % | |
| Cationic | 7.5 mmol | 7.5 mmol | 1:1 | 1, 5, 10 | 1-1.5 | APS/6 |
| Adsorbent | basis | basis | | wt % | wt % | |

### Characterization

Fourier Transform Infrared Spectroscopy (FTIR) was performed to confirm the presence of chemical bonds and therefore expected functionality and conversion in the crosslinked polymer hydrogel composite, and to ensure that no undesired side reactions occurred. A 7000e FTIR spectrometer was used in combination with powdered polymer samples (dried in oven prior to testing) over a range of 700-4000 cm⁻¹ with 32 scans and a resolution of 8 cm⁻¹. A comparison was also made to PEGDA 700 liquid monomer and BSA in its crystalline form. Graphs were trimmed to exclude 0% transmittance values and the x-axis was reversed.

### DI Water Swelling Studies

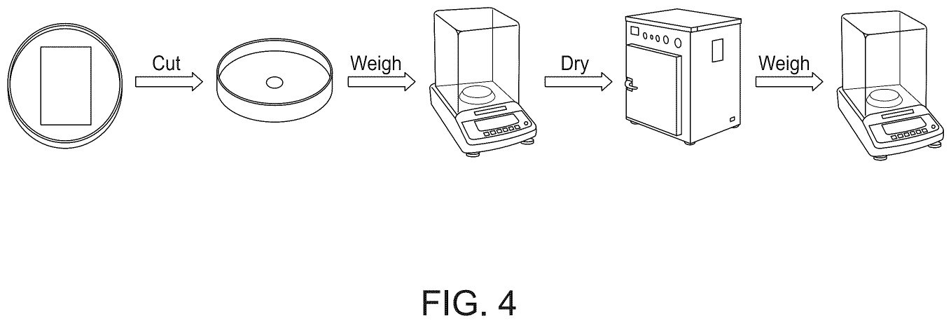

Swelling studies were conducted in DI water to test the swelling capacity of the hydrogel systems (their ability to absorb water). For a standard swelling study, the polymer discs would be cut, washed, dried in an oven and dry masses would be taken. See FIG. 4. They would then be placed in water and the wet (swollen) masses would be taken after some elapsed time in which the polymers would first be patted dry. The swelling ratio (q) would then be calculated according to Equation 2.

$$q = \frac{Wet M ass}{Dry Mass} \qquad ( 2 )$$

Unfortunately, the polymer systems containing DMAPA-Q swelled to the extent that the structural integrity of the polymer fell apart during the study. There is due to stress within the system when the water enters the crosslinked network such that the polymer breaks into smaller pieces to the extent that it becomes difficult to take the wet masses accurately. For this reason, inverse swelling studies were performed for all the systems (for consistency purposes). These were different in that the polymers were cut immediately after forming them and placed in water such that the wet mass was taken first. Consider that at this time, the polymer already contains some water from the recipe. Time zero was then marked while the polymer was in this relaxed state and the wet mass would be taken after some elapsed time. Following this, the discs were placed in the oven at which point the dry masses were taken. The swelling ratio was calculated using the same equation as before and data was taken in triplicate.

### Model Dye Sorption Studies

Model dye sorption studies were conducted to determine the affinity and capacity of the hydrogels. The dyes of choice were indigo carmine and metanil yellow. These were chosen due to a study performed by the EPA which found that indigo carmine (466.4 g/mol) and metanil yellow (375.4 g/mol) performed similarly to PFOS (500.1 g/mol) and PFHxS (perfluorohexane sulfonate) (400.1 g/mol), respectively, when tested with an ion exchange resin due to their similar functional groups and molecular weights.

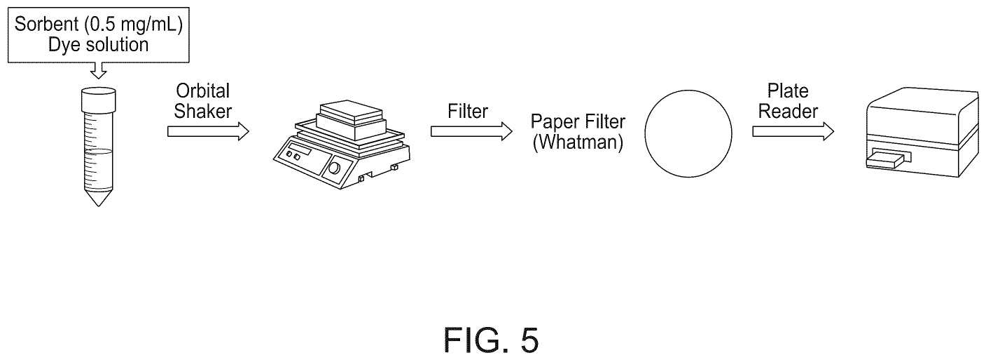

To begin, a dye stock solution was made by adding powdered indigo carmine or metanil yellow to DI water in a glass VWR storage bottle. See FIG. 5. It was then mixed thoroughly by placing the bottle on an orbital shaker. Multiple concentrations of dye were tested until reaching one in which the materials produced a range of removal efficiencies up to 100%. A removal efficiency of 100% would not be ideal because the material may be able to remove more of the dye than is present, and this makes for a less effective comparison between the materials. The peak wavelength at which the concentrations were evaluated were obtained using UV Visible Spectroscopy. These were found to be 610 nm for indigo carmine and 435 nm for metanil yellow. By choosing these wavelengths, the absorbance readings would be the most sensitive to the dye present in solution such that more precise estimates of the resulting concentrations could be obtained. Since both wavelengths were within the visible light spectrum, absorbance readings were taken using a plate reader.

The next step was to develop a calibration curve. A sample was extracted from the dye stock solution and then a series of dilutions were performed using DI water. The dilutions were performed in 1.5 mL microcentrifuge tubes and each time DI water was added for dilution, the solution was mixed before extracting more of the sample for the next dilution. In some cases, multiple samples were retrieved from the stock solution and dilutions were performed on each sample. In other cases, a 5 series serial dilution was performed with one sample which reduced the concentration by half each time. Upon increasing the dye concentrations, it was found that the absorbance readings were too high for the plate reader to quantify, so stronger dilutions were made. To develop the calibration curve, absorbances needed to be obtained for each known concentration/dilution using the plate reader's absorbance function. The principle underlying this is Beer's law as shown in Equation 3 which relates absorbance (A) to dye concentration (c) where l is the path length through which the light passes and £ is the molar absorptivity of the dye solution. Upon plotting the known concentrations against the absorbances, the data could be fit according to a linear trendline with the general format of y=mx+b.

$$A = ε lc \qquad ( 3 )$$

After obtaining the calibration curve, the next step was to weigh out specific masses of dry powder from each polymer being tested and place them in 50 mL centrifuge tubes with the proper amount of dye stock solution such that an intended sorbent dose was obtained (0.5 mg/mL=5 mg sorbent/10 mL stock). The centrifuge tubes were then all allowed to sit for a specified amount of time, not necessarily reaching equilibrium. In conducting the studies for the same amount of time, this allowed for a relative comparison of removal efficiencies. During this time, the tubes were sitting on an orbital shaker often around 200 RPM to prevent the sorbent from settling at the bottom of the tubes. At the end of the elapsed time, the sorbent was filtered out of the tubes. For this, 70 mm Whatman 4 cellulose paper filters were used. The resulting supernatant was then collected in a new 50 mL centrifuge tube. After this, the samples were pipetted into a 96 well plate for absorbance collection.

Ultimately, the calibration curve trendline was used to determine the dye concentrations from the absorbance readings and then the percent removal (removal efficiency) was calculated using the negative control dye concentration as the baseline according to Equation 4. Many studies also included other controls as well, including PAC alone when testing hydrogels which contained PAC. No positive control was included for the BSA systems yet. This method requires further development due to its smaller size and filtration complexities.

$$Percent Removal ( \% ) = \frac{[ Filtered Negative Control ] - [ Sample ]}{[ Filtered Negative Control ]} * 100 \qquad ( 4 )$$

### Results and Discussion

A series of PEGDA-based composite hydrogels were successfully synthesized. These involved varying amounts of PEGDA 700, DMAPA-Q, PAC, and BSA. All polymers were prepared using free radical polymerization and select samples were subsequently characterized using FTIR, DI water swelling studies, and were finally tested according to model dye sorption studies.

### FTIR

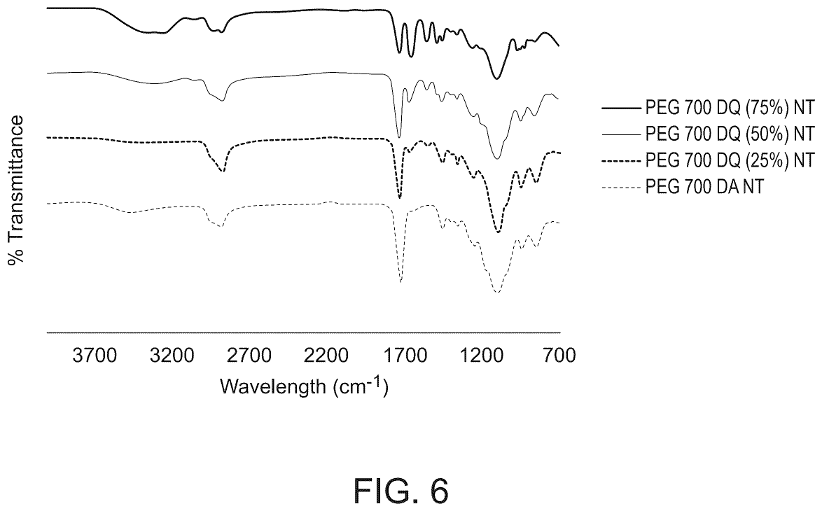

The results of the NT systems are shown in FIG. 6. In general, the expected peaks were observed. These include the C═O peak around 1700 cm⁻¹, the C—H peak around 2900 cm⁻¹, and the N—H peak around 3200 cm⁻¹ for the cationic systems. Note that the C═O peak grows in intensity with decreasing amounts of DMAPA-Q due to the fact that PEGDA contains 2 times as many C═O bonds as DMAPA-Q. Also, as mentioned earlier, the N—H peak is only observed in systems containing cationic as PEGDA lacks this bond. The peak around 3300-3400 cm⁻¹ varies here and is representative of the O—H bond. This may indicate the presence of water in the hydrogels. What's important about this is the fact that the hydrogels are dried prior to testing, so this may suggest that some systems are more hygroscopic than others, perhaps observed with the increasing O—H peak intensity with increasing DMAPA-Q. It may also suggest that some hydrogels were not completely dried, which is the assumption here with PEG 700 DA NT.

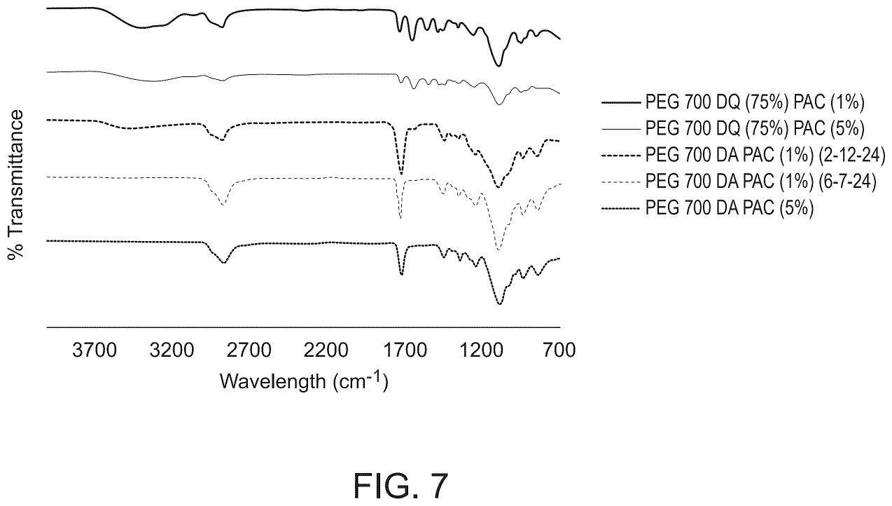

During the previous round of FTIR, the systems containing PAC were also tested. For these, the graphs were quite similar compared to the NT systems. See FIG. 7. The purpose of this FTIR was to analyze the difference between 1 and 5 wt % PAC in the non-cationic and 75% DMAPA-Q systems. The main difference between them appears to be in the peak intensity. In general, the peaks tend to be shallower in the higher wt % PAC systems and this may be due to a lower conversion. Consider that the PAC may physically impeded crosslinking during polymerization which may lead to a higher amount of unreacted material. Also, a comparison between PEG 700 DA PAC (1%) synthesized on Feb. 12, 2024 and Jun. 7, 2024 was made. The concern here was that they had different properties and binding affinities. These may be attributed to the synthesis in which one was left to sit for some time prior to polymerization. There appear to be minor differences between the two, but these may be considered negligible.

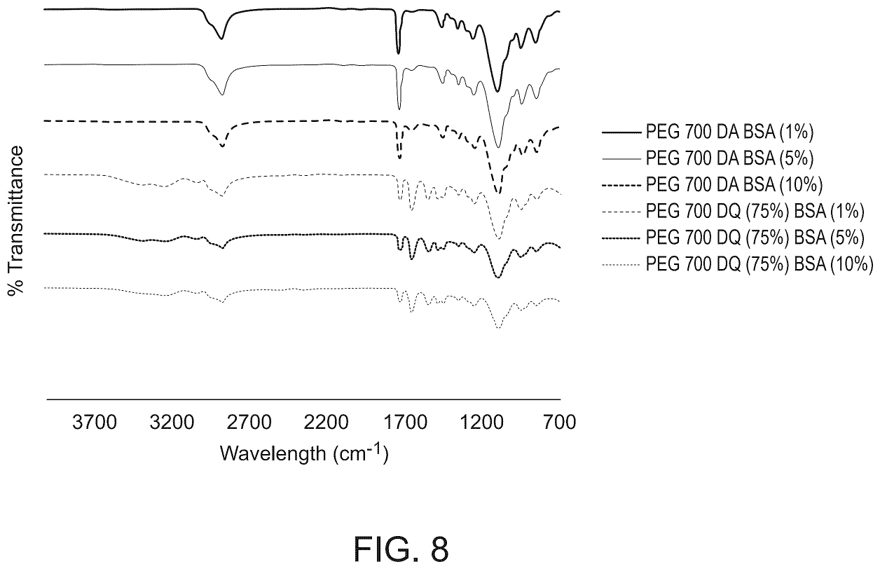

The other adsorbent was BSA and these systems were also tested using FTIR. For these, the same peaks are observed suggesting expected functionalities are present. See FIG. 8. Although no obvious peaks solely due to BSA were initially observed, there may be an increasingly shallow O—H peak with increasing BSA in the 75% DMAPA-Q systems. As mentioned earlier, this may suggest that these systems became decreasingly hygroscopic, which may suggest a lower cationic incorporation and therefore a lower conversion in these high BSA systems.

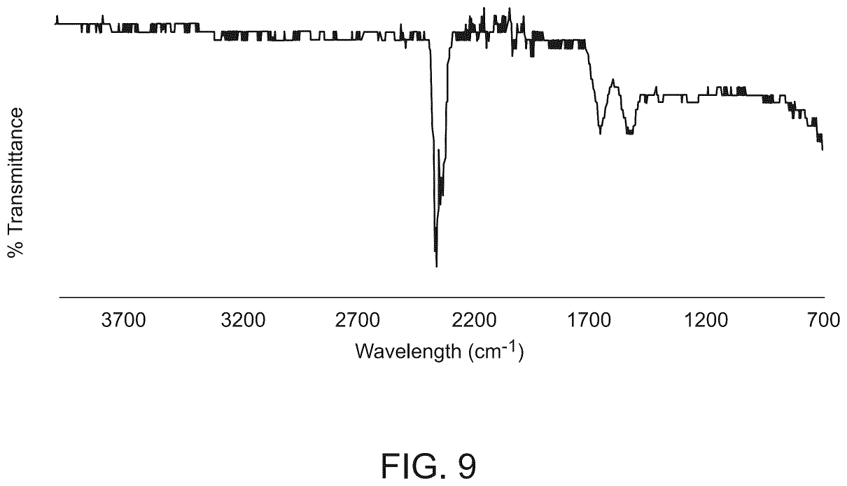

As a control, BSA alone was tested in its solid, crystalline form. The nature of the material made it difficult to obtain a clear graph and peaks, but the graph is shown below in FIG. 9. Although there appears to be a relatively strong peak around 2350 cm⁻¹, its impact on the hydrogel FTIR is unclear.

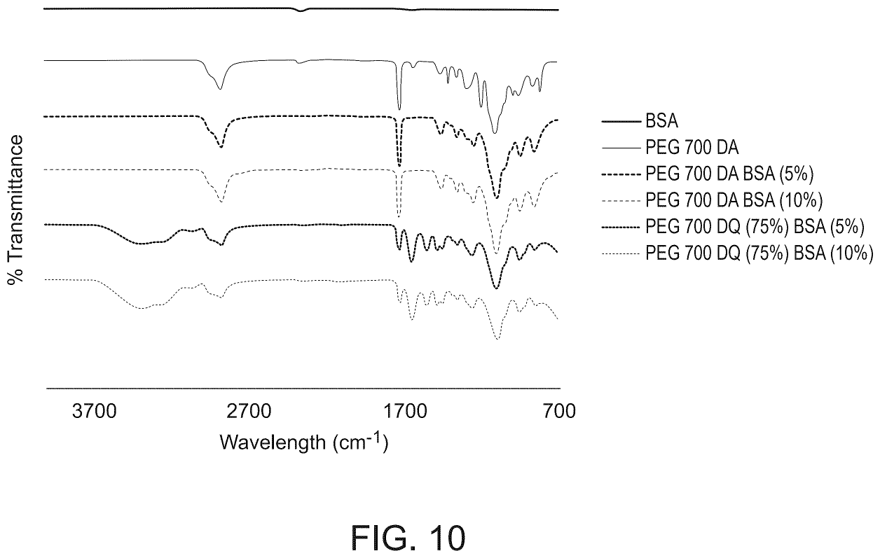

This is especially true when the BSA FTIR is placed on the same scale as the other BSA hydrogels as seen in FIG. 10. For now, the direct impact of BSA appears minimal, which is similar to the PAC hydrogels. Additionally, the O—H peak in the cationic systems appears extra intense. This is primarily because the hydrogels were not allowed sufficient time to dry completely prior to testing. Another difference is that the synthesis method was different than the previous set of BSA hydrogels. These changes don't appear to impact the crosslinking/polymerization significantly when compared to the FTIR in FIG. 8.

### Inverse Swelling Studies

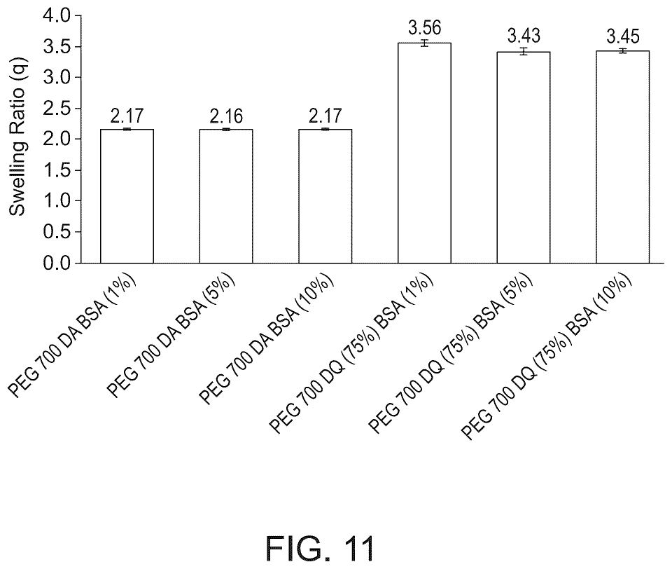

In the following swelling study in particular, the hydrogel was left to sit in DI water for around 2 days and then cut, washed, and finally placed in DI water again for an additional 19 hours. This helps to ensure that the obtained swelling ratios are at equilibrium. The results of this inverse swelling study are shown in FIG. 11.

The three samples on the left are non-cationic and those on the right are cationic (75% DMAPA-Q). Notice that the swelling ratios for all the non-cationic systems are within error of each other despite the variation in wt % BSA. This suggests that the BSA has minimal impact on the swelling properties. On the other hand, with the cationic systems, an increase in BSA led to perhaps a minor decrease in swelling capacity. This is similar to PAC systems tested in the past and it may be due to multiple reasons. For one, the BSA physically occupies space in the crosslinked network. It may also be due to the BSA decreasing the mass conversion and therefore lowering the DMAPA-Q content which has been found to have the largest impact on the hydrogel's swelling ability. Additionally, this suggests that there is additional BSA incorporation in the 5 and 10 wt % BSA systems relative to the 1 wt % BSA system; however, there may be limited further incorporation in the 10 wt % BSA system relative to the 5 wt % BSA system as seen in the similar swelling ratios which are within error of each other. The same thought process may be applied to the non-cationic systems as well to suggest that the BSA is not effectively incorporated in the non-cationic systems, especially at higher BSA levels.

### PFAS Model Dye Swelling Studies

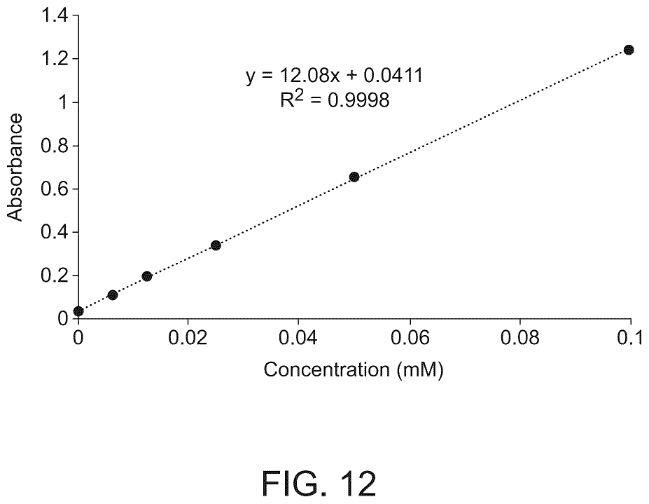

In previous studies, it was observed that the PAC systems did not experience higher levels of sorption relative to their analogous NT systems. To test this, a sorption study was conducted. The dye stock concentration was set at 0.1 mM and the calibration curve was developed using a 5 series serial dilution with DI water at the end. See FIG. 12.

The test for PAC accessibility within the crosslinked network was conducted using a non-cationic system with and without PAC (1 wt %). The sorption study was then allowed to run for 20 hours and for this study only, Whatman 5 filters were used instead (much smaller pore size). Results showed that the removal efficiency with PAC was much higher (nearly a 4-fold increase) relative to its NT counterpart. This suggests that PAC is in fact accessible by the indigo carmine within the crosslinked network, at least in non-cationic systems.

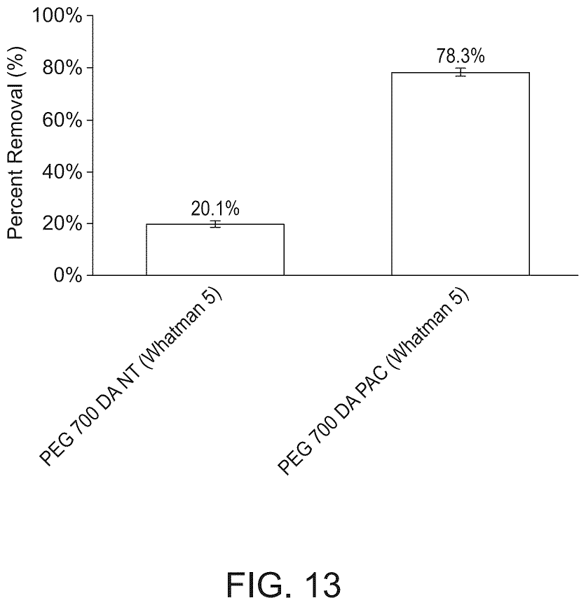

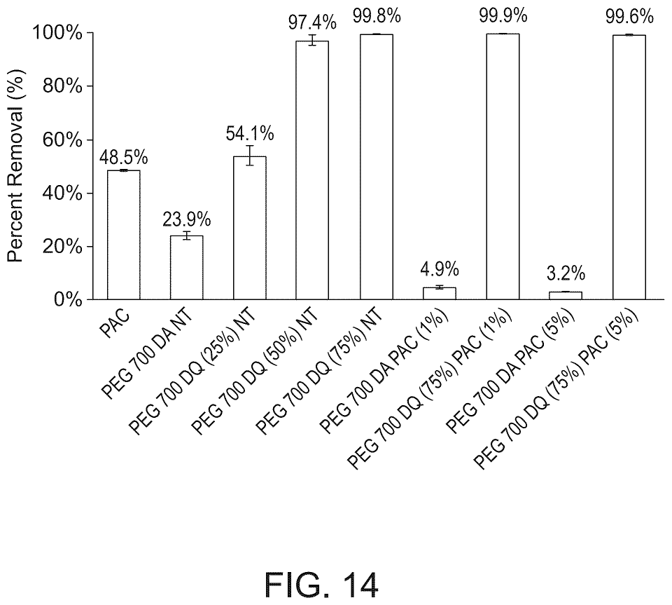

In the past, a starting concentration of 0.1 mM indigo carmine found ˜100% removal for cationic systems, so the initial concentration was doubled (0.2 mM) and a new sorption study was conducted. Results showed that increasing DMAPA-Q led to increased removal efficiency (predominantly due to an assumed electrostatic affinity with the anionic dye). See FIG. 14. Additionally, PAC showed good removal and PEG 700 DA NT alone had some removal. As with the 0.1 mM dye, the 50% and 75% DMAPA-Q systems experienced nearly 100% removal as well, independent of the varying amount of PAC (1 and 5 wt %). Outliers included PEG 700 DA PAC (1% and 5%). As mentioned earlier, there were concerns with the elapsed time prior to polymerization when synthesizing certain hydrogels and these two were included. The properties of the hydrogel may have been impacted such that the true removal efficiency was not well represented as these results were inconsistent with the previous sorption study in FIG. 13.

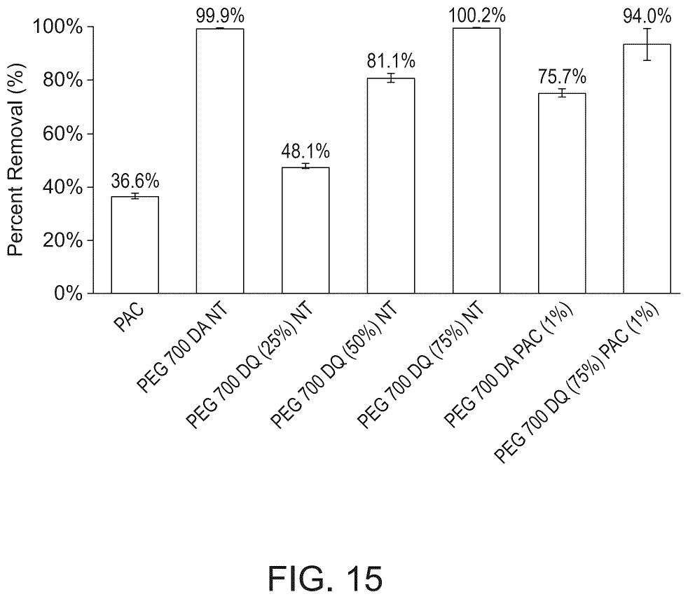

Again, the starting indigo carmine concentration was increased, this time to 0.4 mM. Note that this study was run for longer (46 hours). See FIG. 15. The same pattern with increasing DMAPA-Q and increasing removal efficiency was observed; however, new trends were observed as well. For PEG 700 DA NT, the resulting supernatant was yellow. This could have been due to an unintended degradation of the dye over the course of the study as the study's duration was more than doubled. Therefore, these results were not reliable. Additionally, the PEG 700 DA PAC (1%) system now saw a higher level of sorption which was closer to the expected value as determined by the study in FIG. 13. Still, 100% removal was observed in the 75% DMAPA-Q system.

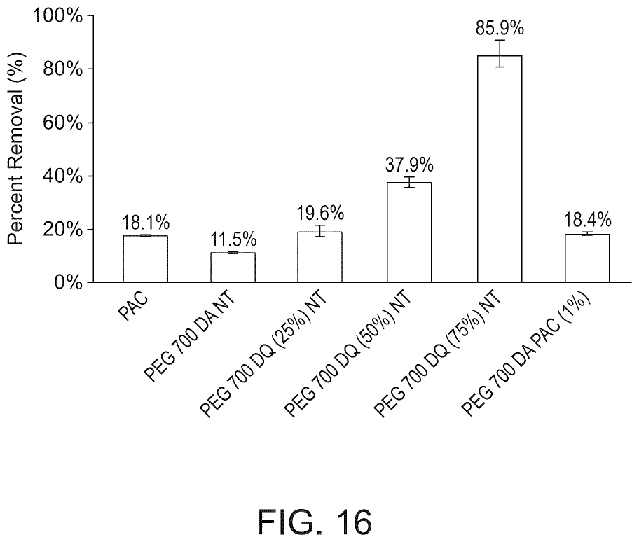

This time, the initial concentration was increased to 0.8 mM indigo carmine. See FIG. 16. With this study, PEG 700 DA PAC (1%) exhibited the higher expected removal efficiency relative to its NT counterpart. This removal was quite similar to PAC alone (positive control). Finally, the increasing removal trend with DMAPA-Q reached a maximum 85.9% removal which was below 100% as desired. This signifies that the sorbent removed as much of the dye from solution as it was able to. Ultimately, the highest removal efficiency in this study was seen with PEG 700 DQ (75%) NT which at a 0.5 mg/mL sorbent dose demonstrated ˜86% removal over 20 hours with an initial 0.8 mM indigo carmine concentration.

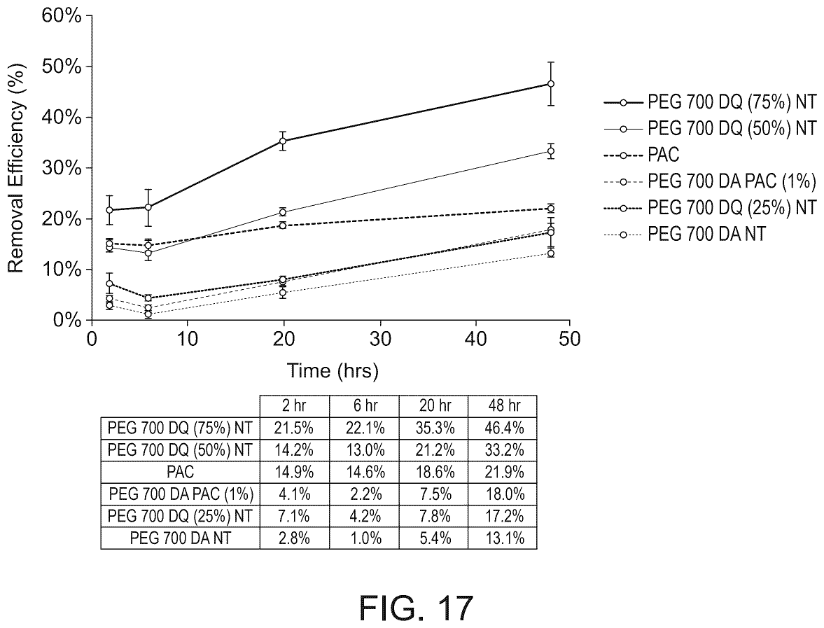

After finding the optimal starting dye concentration, a kinetic study was carried out. See FIG. 17. This study was carried out similarly to equilibrium studies, except that sorbent was weighed out the day prior to beginning the study, and that 15 mL centrifuge tubes were used instead of 50 mL tubes. Also, the start and end times varied for the different timepoints. Ultimately, the removal efficiency for all systems increased over time as expected, except for a slight decrease in removal from the 2 hour to 6 hour timepoint. This may be due to the fact that the start times were staggered. This could have impacted the amount of time in which the sorbent was exposed to moisture in the air (in the tube) and therefore impacted the removal kinetics at these lower timepoints. With the 20 and 48 hour timepoints, this discrepancy may have been diminished. When compared to the previous 20 hour equilibrium study, the same timepoint produced much lower removal efficiencies and this difference is also attributed to the elapsed time between the time the sorbent was removed from the oven and when the dye was added. Only the PAC (positive control) had a similar removal at this time point, but PAC is normally stored outside of the oven so this would have no real impact on the kinetic results. Finally, there appears to be more sorption beyond 20 hours as the removal increases at 48 hours. This indicates that equilibrium is not yet met at 20 hours and may not be reached by 48 hours either. This is why the previous studies are all run at 20 hours. This allows for their relative removal efficiencies to be compared without making definitive statements about their reaching equilibrium.

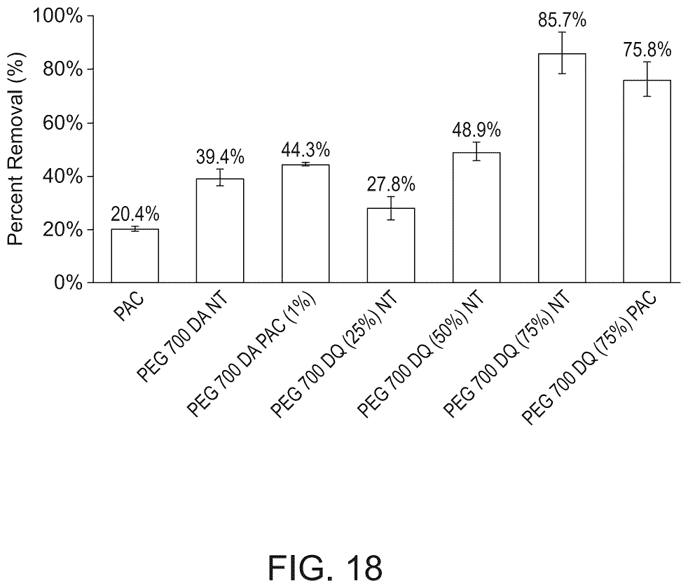

Next, another sorption study was run at 48 hours with the same starting indigo carmine concentration as before (0.8 mM) to reassess the removal at this final kinetic study timepoint. With this, the sorbent was removed from the oven directly before adding the dye solution as normal. Results showed that the removal for all of the sorbents (aside from PAC) were higher than in the kinetic study at 48 hours. See FIG. 18. This suggests that error was introduced during the kinetic study and that the data may be unreliable. Additionally, when compared to the 20 hours study, near all of the sorbents demonstrated increased removal. PAC exhibited the smallest increase in removal, whereas PEG 700 DO (75%) NT demonstrated no increase in removal. This suggests that its removal kinetics are faster which may prove to be beneficial in real-life applications.

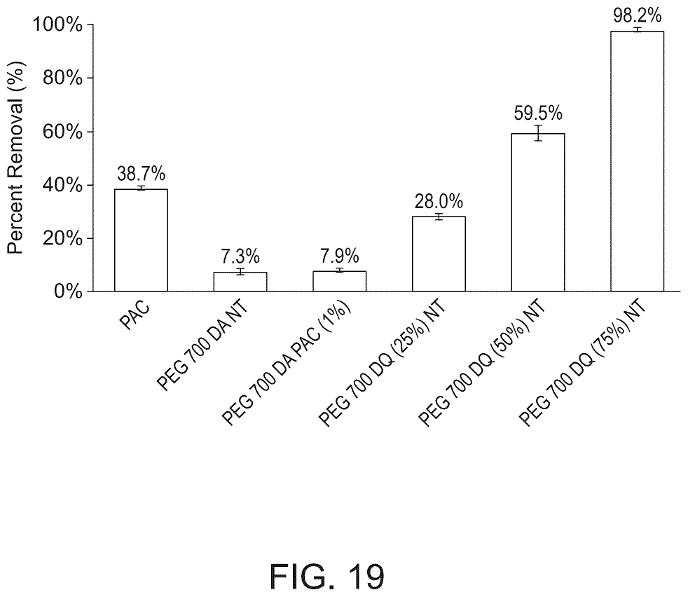

At this point, the dye of interest was switched to metanil yellow at the same initial concentration (0.8 mM). See FIG. 19. This study demonstrated similar trends to the indigo carmine sorption study at 20 hours, except that the removal efficiencies of PEG 700 DA NT and PAC (1%) were lower and those of the PAC and PEG 700 DQ (25, 50, 75%) NT were all higher. This suggests that PEGDA has a lower affinity for metanil yellow. It also suggests that the cationic systems (and PAC) may have a higher affinity for PFHxS than for PFOS. This could be due to the lower molecular weight and size of metanil yellow compared to indigo carmine and how that relates to its ability to fit within the crosslinked network.

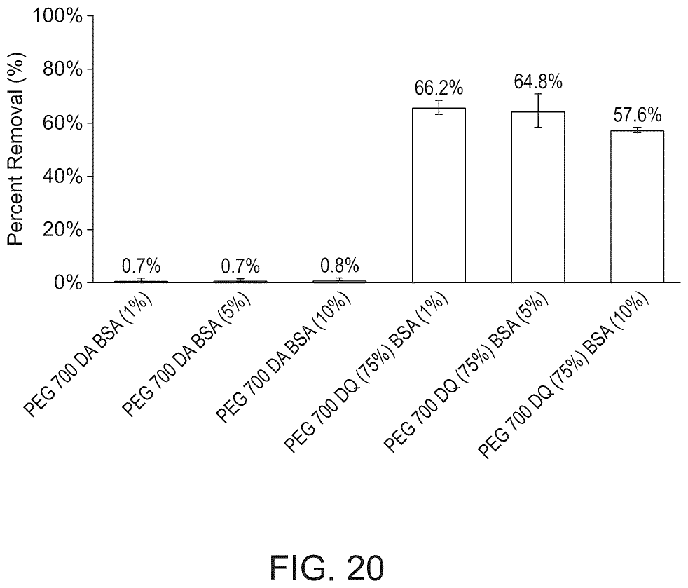

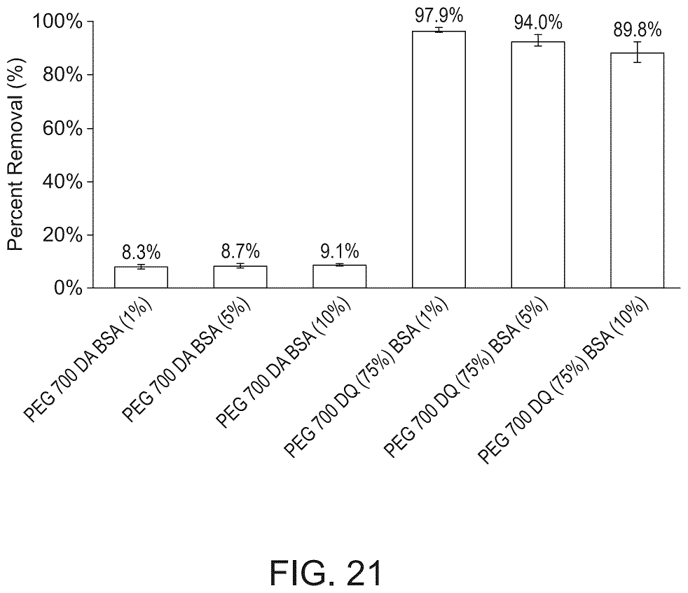

The last couple of studies were focused on testing the newly synthesized BSA hydrogels. For these, both dyes were tested. With the first round of BSA hydrogels, the same synthesis procedure as with the PAC systems was carried out. Results with indigo carmine showed that the removal with non-cationic systems was negligible and that the removal with cationic systems decreased with increasing BSA. See FIG. 20. This suggests that BSA interferes with the ability of the hydrogels to bind the indigo carmine. This was unexpected and suggested that the BSA may have been denatured and that this causes the BSA to lose its PFAS affinity. It may have also impacted the conversion and led to a lower cationic incorporation, thus decreasing the removal further. With the metanil yellow, results showed that removal with non-cationic systems increased slightly with BSA incorporation. See FIG. 21. This is expected based on literature. With the cationic systems, the same trend as the indigo carmine was observed.

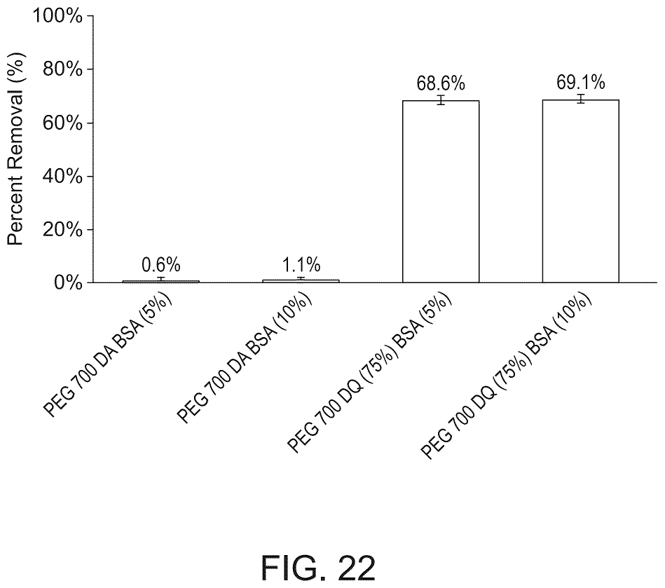

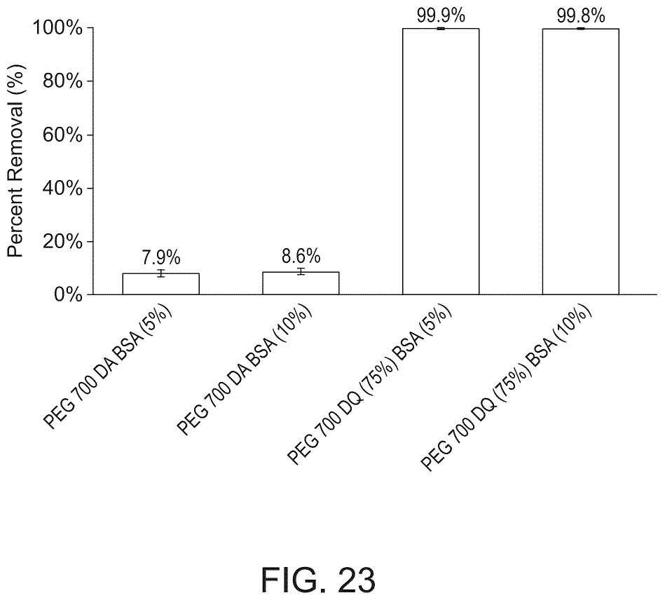

Under the assumption that the BSA was denatured previously, a new set of BSA hydrogels were synthesized (only focused on 5 and 10 wt % BSA this time). The synthesis procedure was modified such that no sonication was performed, the APS was reduced, and the oven temperature was lowered to 40° C. With this, results showed that non-cationic systems still showed negligible removal of indigo carmine and similar removal as before with metanil yellow. See FIGS. 22 and 23. With cationic systems, a higher removal than before was seen with indigo carmine (yet still lower than without BSA), and a higher removal than before was seen with metanil yellow (also higher than without BSA). This suggests that the addition of BSA to the hydrogels may only have a positive impact on the affinity for metanil yellow (PFHxS) using current synthesis methods.

### CONCLUSION

The successful polymerization of a series of various PEGDA based composite hydrogels resulted in the determination of chemical functionalities, swelling ratios, and sorption capacities. The dominant impact across the results appears to be the incorporation of DMAPA-Q as a cationic co-monomer. With the addition of DMAPA-Q, there appears to be an increase in swelling capacity and with sorption, the positive charge introduces a charge-based affinity with the anionic dyes.

Although the method and synthetic-based hydrogels and synthetic-based hydrogel composites of this disclosure have been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

What is claimed:

1. A method for enterosorptive removal of a target molecule from a digestive tract of an individual, comprising orally administering to the patient a synthetic-based hydrogel or synthetic-based hydrogel composite incorporating a hydrogel base material and a functional co-monomer, wherein the synthetic-based hydrogel or synthetic-based hydrogel composite is adapted to bind and remove the target molecule from the digestive track without being metabolized or absorbed into systemic circulation.

2. The method of claim 1, wherein the hydrogel base material is selected from a first group of materials consisting of poly(ethylene glycol), poly(acrylamide), poly(hydroxyethyl methacrylate), poly(acrylic acid), poly(methacrylic acid), poly(N-isopropyl acrylamide) and combinations thereof.

3. The method of claim 2, wherein the functional co-monomer is selected from a second group of materials consisting of a co-monomer with cationic functionalities, a co-monomer with anionic functionalities, a co-monomer with Zwitterionic functionalities, a co-monomer with aromatic functionalities, a co-monomer with hydrophobic functionalities, and combinations thereof.

4. The method of claim 3, wherein the composite additive is selected from a third group of materials consisting of a hydrophobic particulate, a hydrophobic polymer, an ionic particulate, a biological polymer, an ionic polymer, a cationic particulate, a cationic polymer, an anionic particulate, an anionic polymer, a Zwitterionic polymer, a Zwitterionic particulate, a protein, a carbohydrate, deoxyribonucleic acid (DNA), ribonucleic acid (RNA), and combinations thereof.

5. The method of claim 3, wherein the composite additive is selected from a third group of materials consisting of an activated carbon, a powdered activated carbon, a granular activated carbon, an extruded activated carbon, an impregnated activated carbon, and combinations thereof.

6. The method of claim 3, wherein the composite additive is selected from a third group of polymer materials consisting of poly(styrene), poly(methyl methacrylate), poly(ethylene), polypropylene, poly(vinyl chloride), polytetrafluoroethylene, poly(dimethylsiloxane), polyester, polyurethane, poly(vinylidene fluoride), a fluoropolymer, and combinations thereof.

7. The method of claim 3, wherein the composite additive is selected from a third group of polymer materials consisting of poly(lysine), poly(ethyleneimine), chitosan, cationic cellulose, poly(acrylic acid), poly(methacrylic acid) and combinations thereof.

8. The method of claim 3, wherein the composite additive is selected from a third group of materials consisting of a lectin, a DNA binding protein, an RNA binding protein, an albumin, a carbohydrate, and combinations thereof.

9. The method of claim 1, further including binding and removing (a) carcinogens, (b) carcinogen precursors, (c) nitrosamines, (d) nitrates or (e) combinations thereof from the digestive track.

10. The method of claim 1, further including binding and removing a per-fluoroalkyl substance or a poly-fluoroalkyl substance from the digestive track.

11. The method of claim 1, further including binding and removing a dye or a synthetic food dye from the digestive track.

12. The method of claim 1, further including orally administering the hydrogel composite at a dosage rate of between 0.002 to 1.0 grams at a time interval of every four hours to once a month.

13. The method of claim 1, including using poly(ethylene glycol) diacrylate (PEGDA) as the hydrogel base material, using (3-acrylamidopropyl)trimethylammonium chloride (DMAPA-Q) as the functional co-monomer, and using activated carbon as the composite additive.

14. A hydrogel system, comprising:
a synthetic-based hydrogel or synthetic-based hydrogel composite incorporating a hydrogel base material and a functional monomer, wherein the synthetic-based hydrogel or synthetic-based hydrogel composite is adapted to bind and remove the target molecule from the digestive track without being metabolized or absorbed into systemic circulation.

15. The hydrogel system of claim 14, wherein the hydrogel base material is selected from a first group of materials consisting of poly(ethylene glycol), poly(acrylamide), poly(hydroxyethyl methacrylate), poly(acrylic acid), poly(methacrylic acid), poly(N-isopropyl acrylamide) and combinations thereof.

16. The hydrogel system of claim 15, wherein the functional co-monomer is selected from a second group of materials consisting of a co-monomer with cationic functionalities, a co-monomer with anionic functionalities, a co-monomer with Zwitterionic functionalities, a co-monomer with aromatic functionalities, a co-monomer with hydrophobic functionalities, and combinations thereof.

17. The hydrogel system of claim 16, wherein the composite additive is selected from a third group of materials consisting of a hydrophobic particulate, a hydrophobic polymer, an ionic particulate, a biological polymer, an ionic polymer, a cationic particulate, a cationic polymer, an anionic particulate, an anionic polymer, a Zwitterionic polymer, a Zwitterionic particulate, a protein, a carbohydrate, deoxyribonucleic acid (DNA), ribonucleic acid (RNA), and combinations thereof.

18. The hydrogel system of claim 16, wherein the composite additive is selected from a third group of materials consisting of an activated carbon, a powdered activated carbon, a granular activated carbon, an extruded activated carbon, an impregnated activated carbon, and combinations thereof.

19. The hydrogel system of claim 16, wherein the composite additive is selected from a third group of polymer materials consisting of poly(styrene), poly(methyl methacrylate), poly(ethylene), polypropylene, poly(vinyl chloride), polytetrafluoroethylene, poly(dimethylsiloxane), polyester, polyurethane, poly(vinylidene fluoride), a fluoropolymer, and combinations thereof.

20. The hydrogel of claim 16, wherein the composite additive is selected from a third group of polymer materials consisting of poly(lysine), poly(ethyleneimine), chitosan, cationic cellulose, poly(acrylic acid), poly(methacrylic acid) and combinations thereof.
